(19) United States
(12) Patent Application Publication
(10) Pub. No.: US 2025/0292888 A1
(43) Pub. Date: Sep. 18, 2025

(54) MEDICAL DATA PROCESSING APPARATUS AND MEDICAL DATA PROCESSING METHOD

(71) Applicant: CANON MEDICAL SYSTEMS CORPORATION, Otawara-shi (JP)

(72) Inventors: Patrick SCHREMPF, Edinburgh (GB); Alison O'NEIL, Edinburgh (GB); Pavlos PAPAGEORGIOU, Edinburgh (GB)

(73) Assignee: CANON MEDICAL SYSTEMS CORPORATION, Otawara-shi (JP)

(21) Appl. No.: 19/010,722

(22) Filed: Jan. 6, 2025

Related U.S. Application Data
(60) Provisional application No. 63/566,556, filed on Mar. 18, 2024.

(51) Int. Cl. G16H 30/20; G06T 7/00; G16H 50/70

(52) U.S. Cl. CPC G16H 30/20; G06T 7/00; G16H 50/70; G06T 2207/20081

(57) ABSTRACT

A medical data processing apparatus for determining performance of a trained model, the medical data processing apparatus comprising processing circuitry that is configured to:
provide to the model an input comprising data in response to which the model provides an output comprising at least one finding;
obtain at least one further finding from processing of further medical data; and
compare the at least one finding and the at least one further finding thereby to determine performance of the model.

### FIELD

Embodiments described herein relate generally to a medical data processing method and medical data processing apparatus for monitoring model performance.

### BACKGROUND

The task of assuring that machine learning models, for instance Artificial Intelligence (AI) algorithms, continue to function to expected standards when they are deployed in the real world is challenging, as data can change over time. If the underlying data distribution shifts but has the same relationship with the output, this may be referred to as data drift. The output of an AI algorithms used to process images may comprise labels describing the contents of the images. If labels change, for instance, new diseases emerge such as COVID-19, thus causing a difference in the relationship between inputs “x” and labels “y”, this may be referred to as concept drift. Additionally, when these drifts occur model performance may improve, worsen or be unaffected. This may be referred to as model drift.

According to some known methods, direct measurement of the performance of an AI algorithm may require ground-truth data, such as expert human predictions, which can be time consuming and expensive to obtain. This ground truth data may comprise image segmentations and image classifications.

Data drift in medical imaging data distributions can take place due to a number of reasons, for example including changes in imaging and imaging technology, changes in population demographics and the types of pathologies found in the data. Within these categories, more specific reasons for data drift include the use of a new scanner or imaging protocol, migration or ageing of a population, man-made and natural disasters and viral outbreaks.

Multi-Modal Concordance (MMC) is a method for measuring the degree of data drift between snapshots of a stream of imaging input data. Statistical methods are used to compare data distributions between a reference set (earlier in time) and a detection window (later in time). The data types considered are images (latent representation from a virtual autoencoder), corresponding image metadata, and a model's predictions. This results in an MMC score that can be plotted over time. Concordance in this context is a measurement of agreement or consistency, such as between data in the reference set and the detection window. It is known that data drift can be measured using input images with corresponding metadata and model predictions based on the input images. Such methods indirectly detect a potential drift in model performance. However, while shifts in data often cause a change in model performance, if a model is robust against the underlying changes, then performance may be unaffected.

Computer aided diagnosis (CAD) comprises technologies that assist doctors and clinical personnel in interpreting medical data, such as medical images. CAD may comprise image analysis (IA) or the analysis of other modalities of medical data. Medical data may also relate to multiple data modalities, for example one or more of text, images, Digital Imaging and Communications in Medicine (DICOM), metadata and clinical data. Natural language processing (NLP) comprises the process of using a machine learning model, such as a neural network, to process data encoded in natural language. The extraction of natural language data from medical reports, such as radiology reports is known and is commonly used to train AI imaging algorithms. These methods may mine radiology reports to assess the presence/absence of the mention of clinical radiological findings. Rules based off-the-shelf labelers may be used to generate labels for this purpose. Deep learning models trained in a fully supervised fashion where data and ground truth are available, such as a standard pre-trained BERT model may be used. The model may be fine-tuned on annotated radiology reports. Deep learning models able to do more general retrieval of medical concepts e.g. dual encoder or generative large language model such as GPT may also be used.

### BRIEF DESCRIPTION OF THE DRAWINGS

Embodiments are now described, by way of non-limiting example, and are illustrated in the following figures, in which:

FIG. 1 is a schematic illustration of a medical data processing apparatus in accordance with an embodiment;

FIGS. 2(a) and 2(b) are illustrations representing a data stream;

FIGS. 3a and 3b are schematic diagrams representing a method for determining the performance of a trained model in accordance with an embodiment;

FIG. 4 is a schematic diagram representing a method according to an embodiment;

FIG. 5 is a schematic diagram representing a method according to an embodiment;

FIG. 6 is a schematic diagram representing a method in accordance with an embodiment;

FIG. 7 is a plot of concordance versus time;

FIG. 8 is a schematic diagram representing a method in accordance with an embodiment; and

FIG. 9 is an illustration of reference and sample windows in a data stream.

### DETAILED DESCRIPTION

According to certain embodiments there is provided a medical data processing apparatus for determining performance of a trained model, the medical data processing apparatus comprising processing circuitry that is configured to:

provide to the model an input comprising data in response to which the model provides an output comprising at least one finding;
obtain at least one further finding from processing of further medical data; and
compare the at least one finding and the at least one further finding thereby to determine performance of the model. The further medical data may comprise text data.

According to certain embodiments there is provided a computer-implemented method for determining performance of a trained model, comprising:
providing to the model an input comprising data in response to which the model provides an output comprising at least one finding;
obtaining at least one further finding from processing of further medical data; and
comparing the at least one finding and the at least one further finding thereby to determine performance of the model. The further medical data may comprise text data.

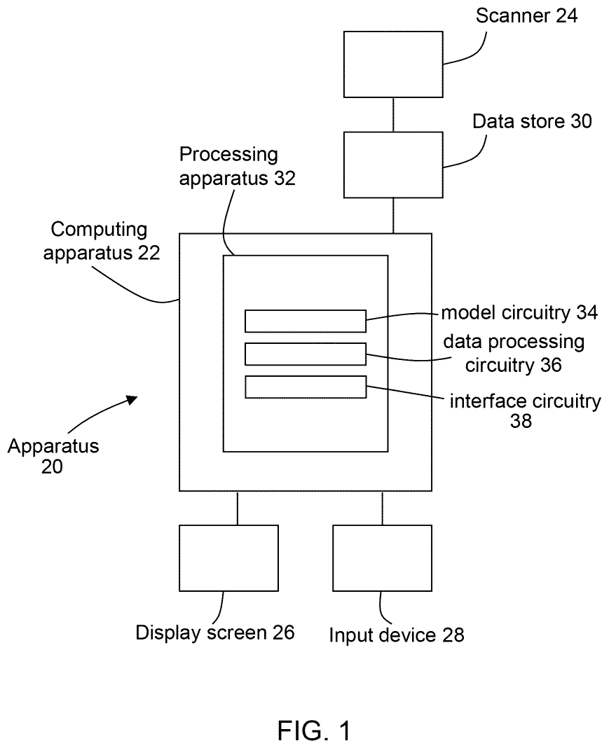

A medical data processing apparatus 20 according to an embodiment is illustrated schematically in FIG. 1. In the present embodiment, the medical data processing apparatus 20 is configured to process medical data. In other embodiments, the medical data processing apparatus 20 may be configured to process any other appropriate data.

The medical data processing apparatus 20 comprises a computing apparatus 22, which in this case is a personal computer (PC) or workstation. The computing apparatus 22 is connected to a display screen 26 or other display device, and an input device or devices 28, such as a computer keyboard and mouse.

The computing apparatus 22 is configured to obtain data sets from a data store 30. The data sets have been obtained or generated using any suitable apparatus or from any suitable source.

In some embodiments, at least some of the data can include, or can be determined from medical imaging data, for instance obtained using a scanner 24. The scanner 24 may be configured to generate medical imaging data, which may comprise two-, three- or four-dimensional data in any imaging modality. Therefore, the scanner 24 is same as imaging modality. For example, the scanner 24 may comprise a magnetic resonance (MR or MRI) scanner, CT (computed tomography) scanner, cone-beam CT scanner, X-ray scanner, ultrasound scanner, PET (positron emission tomography) scanner or SPECT (single photon emission computed tomography) scanner. The medical imaging data may comprise or be associated with additional conditioning data, which may for example comprise non-imaging data.

The computing apparatus 22 may receive data from one or more further medical data stores (not shown) instead of or in addition to data store 30. For example, the computing apparatus 22 may receive medical image data from one or more remote data stores (not shown) which may form part of a Picture Archiving and Communication System (PACS) or other information system.

Computing apparatus 22 provides a processing resource for automatically or semi-automatically processing the data. Computing apparatus 22 comprises a processing apparatus 32. The processing apparatus 32 comprises model circuitry 34 configured to train or otherwise obtain one or more trained models; data processing circuitry 36 configured to apply trained model(s) and to perform other processes; and interface circuitry 38 configured to obtain user or other inputs and/or to output results of the data processing.

In the present embodiment, the circuitries 34, 36, 38 are each implemented in computing apparatus 22 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 22 also includes a hard drive and other components of a PC including RAM, ROM, a data bus, an operating system including various device drivers, and hardware devices including a graphics card. Such components are not shown in FIG. 1 for clarity.

The medical data processing apparatus 20 of FIG. 1 is configured to perform methods as illustrated and/or described in the following.

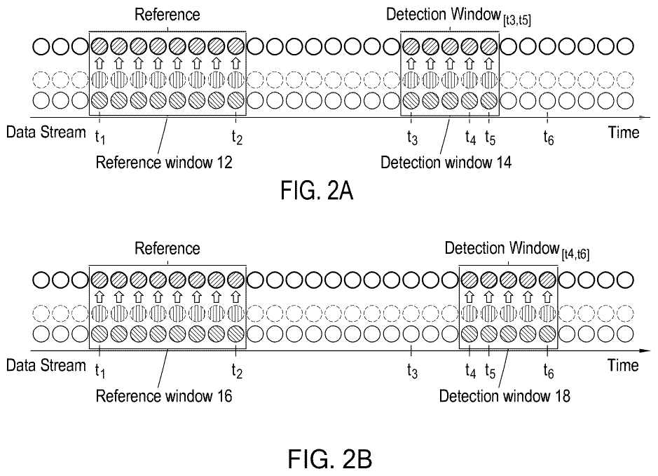

FIGS. 2(a) and 2(b) show two data streams comprising medical imaging data as a function of time wherein time is plotted on the horizontal axis. FIG. 2(a) shows a reference window 12 selecting a reference set of imaging data spanning time t1 to t2 and a later (in time) detection window 14 spanning time t3 to t5 selecting a set of medical imaging data in order to assess data drift between the reference window 12 and detection window 14. FIG. 2(b) shows a reference window 16 spanning time t1 to t2 and a later (in time) detection window 18 spanning time t4 to t6 selecting a set of medical imaging data in order to assess data drift between the reference window 16 and detection window 18 according to certain embodiments. Alternatively or additionally, the data streams may represent the output of a model over a period of time in embodiments. Each window then corresponds to a set of reference input imaging data and a set of detection imaging data provided to the model. Either or both of these configurations, or any number of other configurations may be used to select reference and detection windows for analyzing data drift. Metrics, such as MMC score and probability distributions may be compared between the reference set of data and the data within the detection window. Multiple scores for the same detection window may be averaged to generate a single score for a point in time, a process also known as bootstrapping. In some implementations, the Kolmogorov-Smirnov test can be used to compare continuous distributions resulting from a processing of the data acquired from the reference and detection windows, while the Chi-squared test may be used for categorical variables.

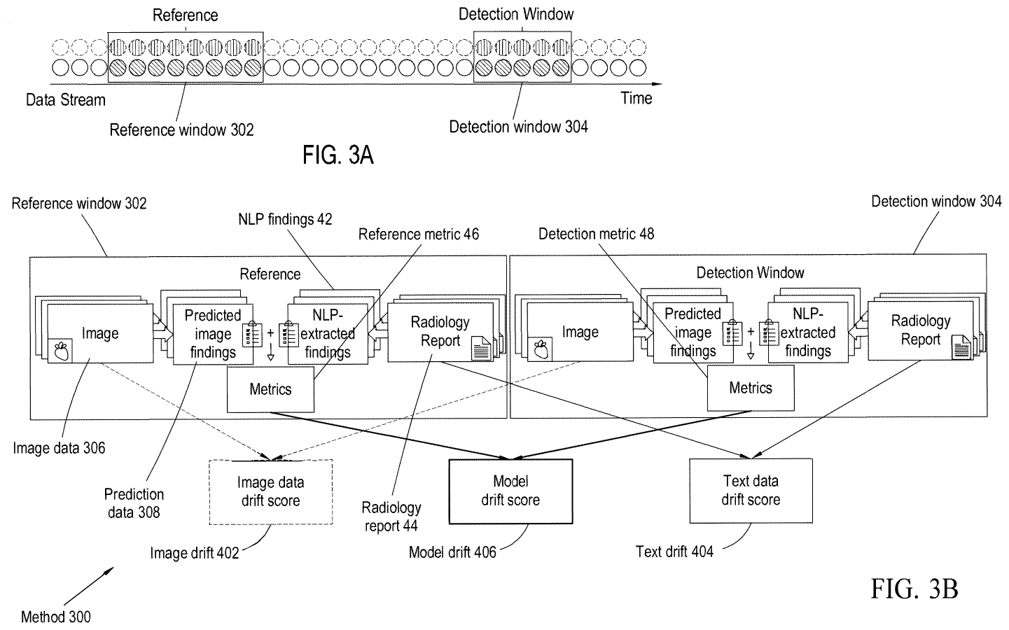

FIGS. 3a and 3b are schematic diagrams representing a method, performed for example by processing circuitry 36, for determining the performance of a trained model in accordance with an embodiment. The model may comprise a trained machine learning model, and is a trained neural network in the present embodiment. In the current embodiment, the data applied to the model includes imaging data and the model may be referred to as an imaging model.

FIG. 3(a) illustrates a data stream at the output of the imaging model plotted against time on the horizontal axis. Each circle in FIG. 3(a) represents a portion of the output data of the model and/or data derived from the output data. The circles can also be used to represent data input to the or a model. FIG. 3(a) also shows a reference window 302 and a detection window 304 that cover a certain number of data samples over a period of time.

The reference window 302 and the detection window 304 each span a period of time which may be referred to as the reference time period and the detection time period respectively. The width of these windows or the time duration that each window comprises and/or the number of data samples in each window may be static, variable or adaptive. In other embodiments, the windows may comprise more or fewer data points in comparison to the illustration in FIG. 3(a). The window sizes of the reference and detection windows may be identical or different. The interval between the detection window and reference window may also be static, variable or adaptive.

In some embodiments, there may be a plurality of reference and/or detection windows. In some embodiments, the reference and detection windows may overlap for some or all of the time duration they are each associated with.

FIG. 3(b) represents a method 300 to measure one or more of the data drift and model drift associated with a model. Reference 302 and detection 304 windows are illustrated in FIG. 3(b) and illustrate the steps of method 300 including calculation of metrics within and between the windows of FIG. 3(a).

Turning to the reference window 302 of FIG. 3(b), medical image data 306 is provided to an imaging model (not shown) and prediction data 308 is received from the output of the model. The prediction data 308 comprises the finding or findings of the imaging model based on its input.

The imaging model may be hosted in the model circuitry 34 or may be hosted in the cloud or other location and be accessible to the model circuitry 34 via a network, such as a wired or wireless communication network.

The circles in FIG. 3(a) depicting data samples may comprise the prediction data 308 of FIG. 3(b).

Medical image data 306 may comprise medical images, for instance obtained using the scanner 24. The scanner 24 may be configured to generate medical imaging data, which may comprise two-, three- or four-dimensional data in any imaging modality. For example, the scanner 24 may comprise an MRI scanner, CT scanner, X-ray scanner, ultrasound scanner or other radiological scanner. The medical imaging data may comprise or be associated with additional conditioning data, which may for example comprise additional data such as metadata, segmentation data or annotation data. The non-imaging data may comprise semantic data associated with the imaging data.

The prediction data 308 is the output of the AI imaging model. The AI imaging model may predict the presence and/or absence of medical features of interest in medical images. The medical features of interest may comprise one or more of anatomical features, pathologies, conditions and other medical features of interest.

The prediction data 308 may comprise semantic data identifying features of interest, for instance, labels identifying anatomical features and pathologies in the medical image data 306. The prediction data 308 may be the output of an image classification and/or image captioning function performed by the imaging model. The prediction data 308 may comprise labels associated with the feature(s) of interest in the medical image data 306. The prediction data 308 may describe the feature(s) of interest in natural language. The prediction data 308 may comprise text data describing pathologies or other conditions and/or text data relating to the presence or absence of pathologies or other conditions in the input data.

One or more, or each, of the images in the medical image data 306 may have an associated medical report which is provided to a natural language processing (NLP) model (not shown) or processed by the application of a natural language process. Sometimes in clinical practice a goal is to report every image or scan that is performed. However, sometimes two or more scans are reported in the same image, and sometimes a report is only created at a later point in time, so it is possible that in some embodiments there are scans with no report at any given point in time.

In FIG. 3(b), the medical report associated with each medical image is a radiology report 44 and contains at least some information and/or data associated with the medical image. The radiology report 44 may contain data associated with medical image data which is not present in the model output. In other embodiments, one or more other medical texts associated with the medical images may serve as or replace the medical report, including patient notes, clinician's reports, patient records and other such reports used in clinical practice.

One or more images in the medical image data 308 may be associated with one or more radiology reports. The report may comprise text, including text in natural language and/or medical image data. The radiology report may comprise annotations of medical image data by a human expert, for instance, generated during routine clinical practice. The NLP model may comprise a machine learning model that is trained on a dataset comprising natural language. The NLP model may be a neural network. Like the imaging model, the NLP model may be hosted in the model circuitry 34 or be hosted remotely while allowing the processing apparatus 32 access to the model using a wired or wireless connection.

The term proxy ground truth may be used to describe labels that are used to calculate metrics, for example to evaluate a model, for example where the labels have been derived from the report in some fashion and have not been created by a human expert directly. In some embodiments, a human expert has created real ground truth annotations (e.g. image annotations—for example highlighting a lesion or other feature in a scan), and/or there may be real ground truth (e.g. report annotations) that can be used to supplement or replace the proxy ground truth. Any reference to proxy ground truth may be replaced by real ground truth for at least some data in embodiments, for data where real ground truth is available.

The NLP model processes the radiology report or reports associated with each input image belonging to the input data 306 to produce NLP findings 42. The NLP findings 42 may comprise a portion of the text in the radiology report 44 or text derived from the radiology report 44. The NLP findings 42 may comprise data obtained by further processing the data contained within the radiology report. NLP findings 42 may comprise semantic data and/or be in the form of natural language. The NLP findings may comprise text data, including labels, describing pathologies or other conditions and/or text data relating to the presence or absence of pathologies or other conditions in the input data. The NLP findings may include attributes associated with the labels describing pathologies and/or conditions wherein the attributes may include laterality, size, condition etc. The NLP findings 42 may be referred to as the ‘proxy ground truth data’. This may be understood by considering that the radiology report 44 comprises data selected using expert human knowledge whereas the imaging model processes data in an automatic fashion without human input. The NLP findings 42 may hence be compared to prediction data 308 for the calculation of reference metrics 46. This comparison may be a computational process and may be carried out using the data processing circuitry 36.

The comparison may comprise a comparison of text labels included in the prediction data 308 and NLP findings 42. The comparison may comprise comparing findings such as labels, semantic data and data associated with the presence or absence of a pathology or condition. The comparison may comprise comparing statistical measures of the performance of the imaging model between the reference and detection/sample windows.

The amount of overlap between the data being compared may be ascertained by the amount of text in one source or data, for instance the NPL findings 42 that matches the text of the prediction data 308. The high overlap between the NLP findings 42 and prediction data 308 may signify a high performance level for the imaging model whereas a low overlap between the NLP findings 42 and prediction data 308 may signify a low performance level for the imaging model.

The reference metrics 46 may comprise one or more indicators of the performance of the imaging model when compared with the proxy ground truth in a reference window 302 or reference time period, including statistical measures of performance.

A similar method to that used for the window 302 is used for the detection window 304. The method followed in the detection window 304 differs from the method of the reference window 302 at least in that the medical image data provided to the imaging model is taken from a detection window 304, or during a detection time period, that is at least partially distinct from the reference time period or the reference window 302. Medical image data is processed using the imaging model while radiology reports associated with the medical image data is processed using the NLP model. The findings of each model are compared to obtain detection metrics 48.

Reference metrics 46 and detection metrics 48 each measure the performance of the imaging model versus the proxy ground truth for the particular window of time that data was used.

In order to measure model drift scores, the reference metrics 46 may be compared to the detection metrics 48. The comparison may comprise comparing statistical measures of the performance of the imaging model between the reference and detection/sample windows. The statistical measure of performance and/or model drift score may represent or may be derived from at least one of F1, Precision, Recall, AUROC, Brier score or other scoring function or comparisons between distributions.

The data processing circuitry 36 may be used to compare the metrics in order to measure model drift associated with the AI imaging model over time and obtain a model drift score 406. The measure of model drift for an AI model may comprise statistical measures such as one or more of p-values, confidence intervals and other such measures.

For the calculation of text drift, the radiology reports associated with the reference window 302 may be compared to the radiology reports associated with the detection window 304 to obtain a text drift score. Similarly, for the calculation of image drift, the medical image data 306 associated with the reference window 302 may be compared to the medical image data associated with the detection window 304 to obtain an image drift score 402. The text drift score 404 and the image drift score 402 may be used to calculate an overall data drift of the system.

While FIG. 3(b) shows only one iteration of the method, this method may be implemented repeatedly or continuously over a period of time wherein performance metrics and/or statistics may be compared for the reference data or findings and the medical image data and predictions over time, thereby to determine a measure of data drift and/or model drift over time.

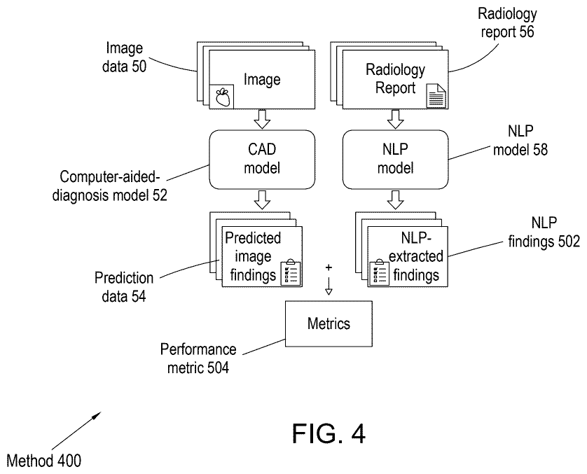

FIG. 4 represents a method 400 of measuring performance metrics of a machine learning model which may be provided separately or in combination with other embodiments. The method 400 illustrated in FIG. 4 is also applicable to data collected in the reference window and the detection window of the method of FIG. 3. Elements of the method that are generally the same as elements of FIG. 3 are not described in detail, for clarity and to avoid repetition. Medical image data 50 is provided to a computer-aided diagnosis (CAD) model 52. The medical image data 50 may comprise medical images wherein the medical images comprise images of anatomical features of interest and/or pathologies. The CAD model 52 may comprise a machine learning model used for processing medical images. Method 400 may be used to measure performance metrics of the CAD model 52. The CAD model 52 is used to obtain findings associated with the medical image data 50 in the form of prediction data 54.

Radiology reports 56 associated with the medical image data 50 are provided to an NLP model 58. The NLP model 58 extracts findings from the radiology report 56 to obtain NLP findings 502. The prediction data 54 and NLP findings 502 are then compared to calculate performance metrics 504 of the CAD model 52. The NLP-extracted findings ‘g’ can be compared with the predicted image findings ‘y’ (output of the computer-aided diagnosis (CAD) model), to produce a set of distributions of performance metrics and statistics ‘P’ wherein P may be defined by:

P(y,g)={\hat{ξ}(y,g)∀ξ∈Ξ}

where Ξ is a set of functions ξ used to calculate performance and is the function applied with bootstrapping. The bootstrapping may comprise averaging multiple scores from one window averaged to generate a single score for a point in time, and/or may be achieved by resampling. The output of these functions may be scores such as F1, Precision, Recall, AUROC, Brier score. Furthermore, there could be separate functions for micro/macro metrics or per-sample/per-label/per-attribute metrics.

Applying method 400 to data collected in a reference window and a detection window results in reference metrics and detection metrics respectively.

Model drift statistics may then be calculated for a detection window d and a reference “r” using proxy ground truth predictions g as

Model drift(y,g,r,d)={φi(Pi(yᵣ,gᵣ),Pi(y_{d},g_{d}))}

where statistical tests φᵢ, such as the Kolmogorov-Smirnov test, the Chi-squared test, weighting, ν, and standardization ‘Γ’ may be used to measure performance statistics. The performance statistics may include p-values and confidence intervals among other such parameters.

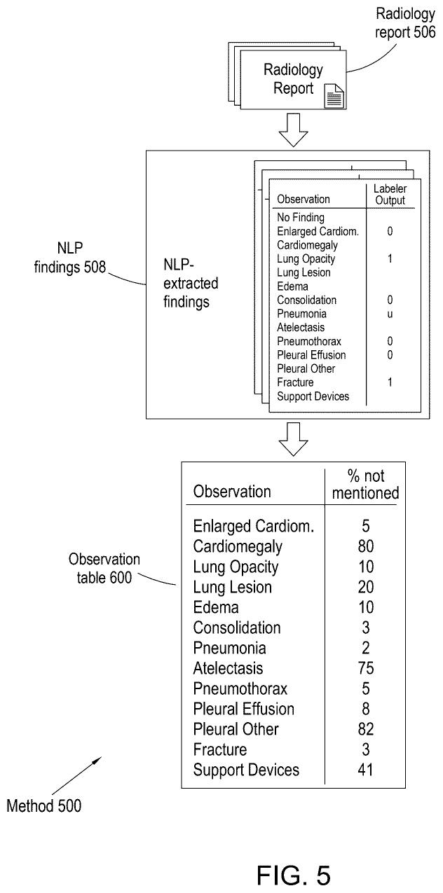

FIG. 5 shows a radiology report 506 and the NLP findings 508 obtained from the NLP model to which the radiology report 506 is provided. Data processing circuitry 36 may be used to generate an observation table 600. The observation table may contain two columns, the first containing a list of observations and the second containing measures of the occurrence of each observation in NLP findings 508. The observations may be labels describing anatomical features and/or pathologies and/or conditions determined by the NLP model. The measure(s) of occurrence may be in the form of absolute numbers, percentages and other such formats. A significant challenge during extraction of proxy ground truth from standard free text radiology reports is that radiology reports can be short, incomplete and sometimes inaccurate. In some embodiments, this may be solved by method 500 in FIG. 5 by ignoring unmentioned pathologies and reporting the number and/or proportion of unmentioned pathologies as a performance metric when measuring the performance of the CAD model. Ignoring unmentioned pathologies and/or anatomical features comprises excluding them from the calculation of a performance metric. Neither the presence or absence of the pathologies and/or features are assumed or determined in such a case. This may be denoted as a function ‘m’ which filters the predictions ‘y’ and proxy ground truth ‘g’:

P(y,g)={\hat{ξ}(m(y,g))∀ξ∈Ξ}

The method may further comprise reporting the proportion and/or percentage of unmentioned pathologies/anatomical features as an NLP-extracted ground truth quality assessment metric when agreement between the machine learning model and the NLP model are reported. The proportion and/or percentage may be presented to the user as an output in addition to any performance metrics presented to the user. The NLP-extracted ground truth quality assessment metric may be used to assess how representative or meaningful at least some performance metrics are, similar to standard deviation or variance or indicating the sample size used (% mentioned). The % not mentioned may be calculated from the outputs of the NLP model, e.g. the NLP-extracted ground truth or proxy ground truth.

In some examples a bias against certain types of imaging findings and certain types of report may be found i.e. the “% not mentioned” will not be randomly and uniformed distributed. The “% not mentioned” might for example indicate if a certain finding is e.g. never reported as present. Causes for this could include potential bias against predicting that finding (for example, perhaps it was underrepresented/not present in the training data).

Follow-up reports can be particularly difficult for a NLP model to extract useful data from, for instance, reports which contain non-specific statements such as “No change since last scan”. For these and other such cases where prior reports are relevant to the data contained in the current reports, the prior reports may additionally be provided to the NLP model for the extraction of findings.

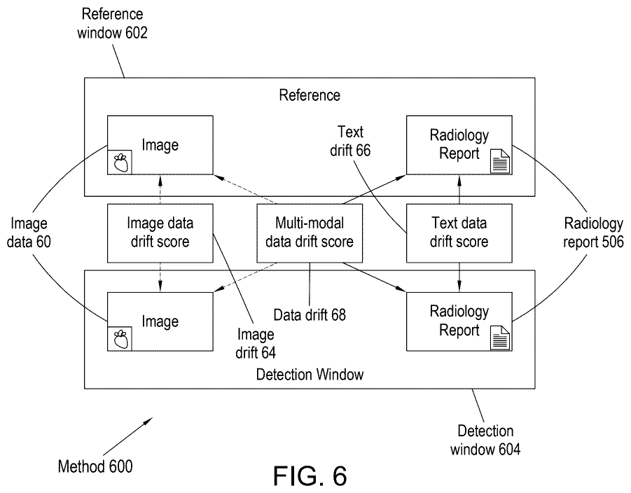

FIG. 6 represents a method 600 of measuring data drift and model drift that may be provided independently or in combination with the other methods disclosed herein. Data distributions may also be compared over time. A shift in data may result in a change in model performance, and alerting users of the machine learning model used for CAD that a change in the data distribution has occurred can aid intervention. The calculation of data drift can be performed using measurements such as Multi-Modal Concordance. This concordance measure may be augmented with the use of multi-modal representations, such as those comprising image and text using Contrastive Language-Image Pre-training (CLIP) and/or Fréchet Inception Distance (FID).

FIG. 6 shows a reference window 602 and a detection window 604. Both windows share the same medical image data 60 and radiology reports 62. Comparison of findings from medical image data between reference and detection windows may be used to calculate an image drift score 64. Comparison of findings from radiological reports between reference and detection windows may be used to calculate a text drift score 66. In embodiments where the medical image data 60 is multimodal and comprises data formats other than images or other than images alone, such as metadata and text, the medical image data may be used to calculate image drift while each other modality may be compared between reference and detection windows to calculate further drift metrics association with these other modalities. Similarly, multi-modal data in radiology reports may also be processed in this way to obtain drift metrics for multiple modalities of data. In some embodiments, distributions or other metrics and/or statistics extracted from the images/text are compared between the reference and detection window to calculate data drift scores.

In some embodiments, a computed Multi-Modal Concordance metric calculated on a detection window d using reference set r may be extended with the additional input of proxy ground truth g to obtain an NLP-informed concordance consisting of the following components:

IDD(d,r)=MMC(dₗ,rₗ)

TDD(d,r)=MMC′(d_{T},r_{T})

MD(y,g,r,d)=φ(P(yᵣ,gᵣ),P(y_{d},g_{d}))

wherein MD is the model drift as described in relation to method 400 of FIG. 4, and MMC is the Multi-Modal Concordance metric. Furthermore, dₗ is the medical image data from the reference set, r_{T} is the text data from the reference set. IDD and TDD are the medical image data drift score and text data drift score respectively.

In some embodiments, the calculation of a text data drift score follows an approach similar to that used to calculate the MMC. In other embodiments, similarity of BERT embedding representations may be used to calculate text data drift. The text data used on such a method comprises the subset of radiology reports for images which were independently reported by radiologists. To compare distributions, for example the distributions from reference and detection windows, statistical tests φ, such as the Kolmogorov-Smirnov or Chi-squared test, may be used. Some embodiments may be used in a setting where images are automatically reported on by a trained model and/or algorithm. In at least some such embodiments, it may be ensures that at least some of the reports are also independently reported on by radiologists or other persons, for example to ensure that the labels extracted from the reports may be used accurately as a proxy ground truth.

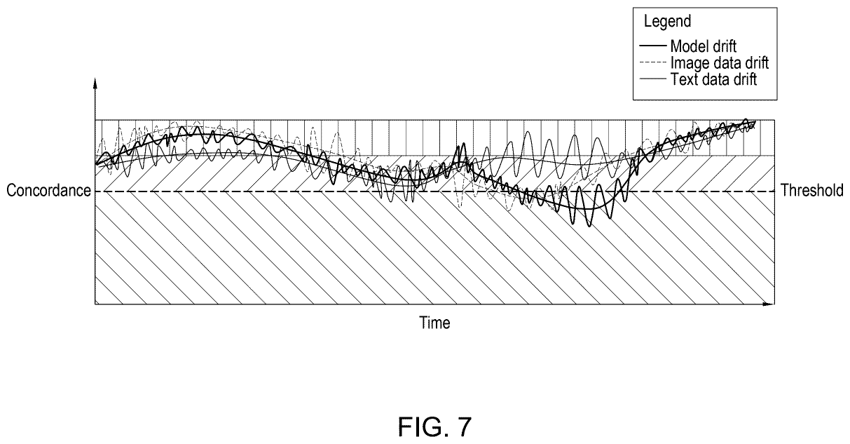

FIG. 7 illustrates how concordance may be plotted and monitored over time. FIG. 7 is a graph of concordance versus time. The graph of FIG. 7 may be displayed in real time using the display screen 26. In FIG. 7, model drift and text data drift and medical image data drift are simultaneously presented allowing an enhanced interpretation of concordance. In other embodiments, a subset of the drifts or any of the derived performance metrics may be represented on the display screen 26. The data is likely to be noisy, as seen in FIG. 7, and so trend analysis over time may be provided. Confidence intervals, for instance, may be used to indicate how much variation is present in a metric at a given time. Each performance metric may have an associated threshold that indicates if a score should be alerted to a user. Metrics may be normalized in order to use a single threshold (as indicated in FIG. 7). The alert may be triggered either by the mean or the confidence interval crossing the threshold, or based on any other statistical parameter related to the data.

The following table analyzes different combinations of concordance related to the model, image and text drift and proposes one or more possible reasons for each situation. In the following table, a value of zero for a drift parameter signifies that the parameter value is below a threshold while a value of one signifies that the parameter value is above a threshold value.

| Model | Image | Text | |
|---|---|---|---|
| drift | drift | drift | Interpretation |
| 0 | 0 | 0 | Model working robustly, everything normal |
| 0 | 0 | 1 | Model working robustly although radiological |
| | | | report contents have changed |
| 0 | 1 | 0 | Model working robustly |
| 0 | 1 | 1 | Model requires investigation - whilst no model |
| | | | drift is detected, there may be concept drift |
| | | | e.g. a new disease has appeared in the images |
| | | | and reports but neither model nor NLP is |
| | | | classifying it |
| 1 | 0 | 0 | Model requires investigation - performance |
| | | | appears to have degraded without underlying |
| | | | data change |
| 1 | 0 | 1 | Model requires investigation - Report content |
| | | | has changed signifying possible concept drift. |
| | | | Unclear whether detected model drift is a true |
| | | | dip in performance or mismatch due to NLP |
| | | | degradation |
| 1 | 1 | 0 | Model requires investigation - image content |
| | | | has changed, possibly due to a change in |
| | | | imaging technology and/or patient population |
| | | | and caused change in model performance |
| 1 | 1 | 1 | Model requires investigation - it appears that |
| | | | both image and text data has changed, |
| | | | suggestion a shift in the distribution of |
| | | | findings causing model performance to drift. |
| | | | This could be because of a change in disease |
| | | | profile. |

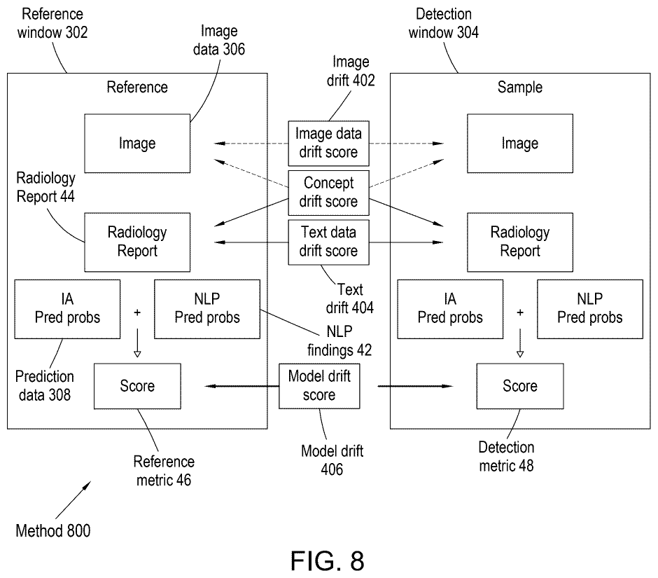

FIG. 8 represents a method 800 for measuring the image, text, model and concept drift scores of a machine learning model used for processing medical image data.

Concept drift may comprise a drift in the mapping between input and output data. A measure of concept drift may represent a likelihood or a probability that a given output results for a given input. In the current embodiment, this may be the likelihood of obtaining a given finding for a given sample of medical image data or radiology report.

In embodiments that use radiology reports and NLP model predictions as a proxy ground truth for the output of an imaging model, monitor the relationship between inputs and outputs can be monitored, and a shift in the mapping between the two can be detected. Concept drift may be measured by monitoring over time the variation in the imaging model outputs obtained from the input data and/or for the NLP findings obtained from processing of radiological reports. The input data and/or the radiological reports used to obtain a measure of concept drift may be the same or different for different time periods.

FIG. 8 shows how each of image, text, model and concept drift scores can be measured for a reference set of data and a detection or sample set of data. FIG. 8 uses the reference numerals used to corresponding items in FIG. 3(b) where these items are common to both figures. Items in detection window 304 that are identical to those in the reference window 302 may be not labelled. FIG. 8 shows a reference window and a detection window. Each window comprises medical image data 306 and associated radiology reports 44. Prediction data 308, which comprises the findings or output of the AI imaging model given medical image data 306 as input is compared to NLP findings, which comprise finding of an NLP model when given radiology reports 44 associated with medical image data 306 as input.

The comparison of the NLP findings 42 and the predictions data 308 is used to obtain the reference metric 46 in the reference window and the detection metric in the detection or sample window. Comparing these metrics between the reference and detection window can be used to generate a model drift score for the imaging model.

Medical image data 306 can be compared between the reference and detection windows to obtain medical image data drift score 402. Text data may be compared between the reference and detection windows to obtain image text drift score 404. Data derived from medical image data using the AI imaging model in the reference window may be compared to data derived from radiology reports using the NLP model in the detection window to measure concept drift. Similarly, data derived from medical image data using the AI imaging model in the detection window may be compared to data derived from radiology reports using the NLP model in the reference window to measure concept drift.

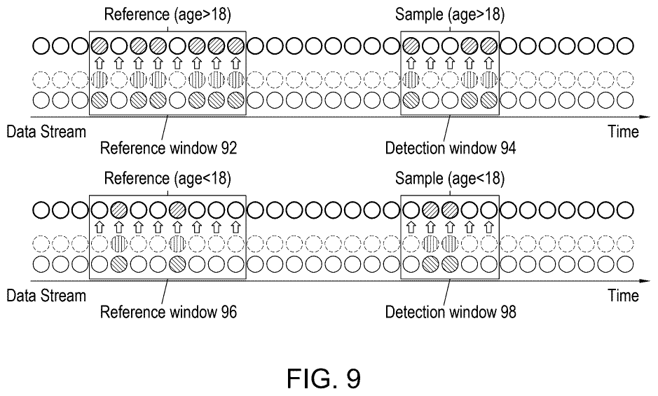

FIG. 9 illustrates how drift scores may be calculated for different subpopulations of data, for instance, based on age of participants. This can provide further information of what type of data drift is occurring and what input data the imaging model's performance is changing based on. This can also aid investigations of bias and fairness of the algorithm that is being analyzed to determine performance.

FIG. 9 shows two data streams. The upper data stream comprises a reference window 92 containing a subset of medical image data as applicable to subjects over the age of eighteen while the sample or detection window 94 also limits to findings related to images from subjects over eighteen years of age. The lower data stream comprises a reference window 96 containing a subset of medical image data as applicable to subjects under the age of eighteen while sample or detection window 98 also limits to findings related to images from subjects under eighteen years of age. In this way, the method can be used to determine measures of model drift, data drift and/or concept drift that are specific to different sub-sets of patients. The sub-sets of patients may, for example, be selected based upon demographic, medical or other patient information.

In some embodiments, the NLP model, imaging model or other model may be used to not only detect the presence or absence of a given finding, but also its attributes (e.g. laterality such as left, right, bilateral), size (small or large etc.), distribution (focal, global), appearance (hyperintense, hypointense) among others. These attributes may be included in the proxy ground truth obtained by the NLP model for comparison with the findings of the AI imaging model and for measuring performance metrics and drift scores.

In various embodiments, there is provided an medical data processing apparatus for determining performance of a trained model, the medical data processing apparatus comprising processing circuitry that is configured to:

provide to the model an input comprising data in response to which the model provides an output comprising at least one finding, wherein optionally the data may comprise medical image data;
obtain at least one further finding from processing of further medical data, wherein optionally the further medical data may comprise text data; and
compare the at least one finding and the at least one further finding thereby to determine performance of the model.

The comparing of the at least one finding and the at least one further finding may be used to determine at least one of a model drift score, a data drift score or a concept drift score.

The input data, optionally the medical image data, may be obtained in respect of a first time period and the performance of the model may comprise performance at the first time period.

The processing circuitry may be further configured to:
determine performance of the model at a different, reference time period, and
compare the model performance at the first time period and the model performance at the reference time period thereby to determine a change of model performance over time.

With regard to the determining of performance of the model at the first time period, the processing of the further medical data may comprise processing the further medical data to obtain proxy ground truth data.

The proxy ground truth data may comprise labels or other findings extracted by processing the text data.

The text data may comprise at least one radiology report, clinician report or patient record.

The processing of the text data may comprise at least one of: applying a natural language process (NLP) to the text data; applying a trained NLP model to the text data.

The processing of the text data may comprise at least one or determining at least one label, or identifying the presence or absence of at least one pathology or condition.

The processing of the text data may comprise assigning an attribute to at least one identified pathology or condition.

The comparing of the model performance at the first time period and the model performance at the reference time period may comprise comparing at least one statistical measure of performance.

The at least one statistical measure of performance and/or model drift score may comprise, represents or is derived from at least one of F1, Precision, Recall, AUROC, Brier score or other scoring function or comparison between distributions.

The performance of the model at the reference time period may be determined by comparing outputs of the model to ground truth data. The ground truth data may comprise annotations of, or findings derived from, medical image data by a human expert, and/or the ground truth data for the reference time period may comprise reference proxy ground truth data obtained by processing further text data for the reference time period.

The comparing of the performance of the model may comprise excluding pathologies that are not mentioned in ground truth data and/or proxy ground truth data in determining the performance of the model.

The processing circuitry may be configured to compare distributions of the medical image data and/or the reference data or findings or representations obtained from the medical image data and/or reference data, over time thereby to determine a measure of data drift over time.

The processing circuitry may be configured to monitor variation over time for the output obtained from the input and/or for the at least one further finding obtained from processing of further medical data, thereby to obtain a measure of concept drift.

The measure of concept drift may represent a likelihood of obtaining a given output, or finding, for a given input, or further medical data.

The input and/or the further medical data used to obtain the measure of concept drift may be either the same or different for different times.

The model drift, data drift and/or concept drift may be determined for selected sub-sets of the input data and/or the further medical data corresponding to sub-sets of a set of patients, thereby to determine measures of model drift, data drift and/or concept drift that are specific to different sub-sets of patients.

The sub-sets of patients may be selected based upon demographic, medical or other patient information.

In various embodiments, there is provided a computer-implemented method for determining performance of a trained model, comprising:
providing to the model an input comprising data in response to which the model provides an output comprising at least one finding, wherein optionally the data may comprise medical image data;
obtaining at least one further finding from processing of further medical data, wherein optionally the further medical data may comprise text data; and
comparing the at least one finding and the at least one further finding thereby to determine performance of the model.

In various embodiments there is provided a method and/or system for monitoring model performance and data drift, comprising
a CAD algorithm which aims to detect a set number of findings,
a NLP model which is trained to extract a representation of the same set of findings as the CAD algorithm from medical records (e.g. radiology reports),
a model performance calculation that compares the outputs of the CAD algorithm and NLP model,
a drift calculation engine that compares the output of the performance calculation over time and outputs a model drift score, and/or
a concordance/drift monitoring system which allows users of the system to view the drift scores over time

There may also be provided a data drift calculation engine that compares the input data over time and outputs a data drift score

The method and/or system may further combine data drift and model drift scores to alert the user to score combinations where the model should be investigated.

An additional calculation and reporting of the number of unmentioned findings in the radiology reports may be provided.

There may also be provided a drift calculation engine that compares the multi-modal data over time and outputs a multi-modal data drift score.

There may also be provided a drift calculation engine that compares the relationships between input and output data over time and outputs a concept drift score.

There may also be provided extension of the drift calculation engines to generate scores for different subpopulations of data.

The CAD algorithm may be replaced by an AI model that does not take medical image data as input (e.g. non-imaging data or multi-modal AI)

Various embodiments can be used to monitor for changes in input data a model, and/or changes in expected output of the model, and/or performance of the model.

A capability may be provided to detect in real time when shifts like data drift and concept drift occur, and further detect whether the model's performance is being affected by these drifts, such as whether model drift is occurring.

In some aspects, a NLP-informed concordance metric is described which leverages expert radiology reports. This approach does not require specialist ground truth such as image segmentations. In some aspects, the approach extracts ground truth from standard text reports such as those that radiologists write in clinical practice. In some aspects, a subset of images is reported independently by radiologists and independently of the model. This NLP-extracted proxy ground truth can then be compared to the model predictions to measure the performance of the model. Additional monitoring of patient data and radiology reports may allow the detection of data drift and model output drift, or model drift. These metrics, compared between a reference window and a detection window, may enable quality assurance of AI algorithms during real-world deployment.

The drift scores can be calculated for different subpopulations of data (e.g. based on age). This can provide further information on what type of data drift is occurring and what data the model's performance is changing on. This can also aid investigations on bias and fairness of the AI algorithm that is being assured.

Features in one aspect or embodiment may be combined with features in any other aspect or embodiment in any appropriate combination. For example, medical data processing apparatus features may be provided as method features and vice versa.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed, the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical data processing apparatus for determining performance of a trained model, the medical data processing apparatus comprising processing circuitry that is configured to:
provide to the model an input comprising medical data, from an imaging modality, in response to which the model provides an output comprising at least one finding;
obtain at least one further finding from processing of further medical data; and
compare the at least one finding and the at least one further finding thereby to determine performance of the model.

2. An medical data processing apparatus according to claim 1, wherein at least one of:
the medical data comprises medical image data generated by the imaging modality;
the further medical data comprises text data.

3. A medical data processing apparatus according to claim 1, wherein the comparing of the at least one finding and the at least one further finding is used to determine at least one of a model drift score, a data drift score or a concept drift score.

4. A medical data processing apparatus according to claim 1, wherein the input medical data is obtained at a first time period and the performance of the model is performance at the first time period, and the processing circuitry is further configured to
determine performance of the model at a different, reference time period, and
compare the model performance at the first time period and the model performance at the reference time period thereby to determine a change of model performance over time.

5. A medical data processing apparatus according to claim 4, wherein with regard to the determining of performance of the model at the first time period, the processing of the further medical data comprises processing the further medical data to obtain proxy ground truth data.

6. A medical data processing apparatus according to claim 5, wherein the proxy ground truth data comprises labels or other findings extracted by processing the text data.

7. A medical data processing apparatus according to claim 1, wherein the text data comprises at least one radiology report, clinician report or patient record.

8. A medical data processing apparatus according to claim 1, wherein at least one of:
the processing of the text data comprises at least one of: applying a natural language process (NLP) to the text data; applying a trained NLP model to the text data; or
wherein the processing of the text data comprises at least one or determining at least one label, or identifying the presence or absence of at least one pathology or condition.

9. A medical data processing apparatus according to claim 8, wherein the processing of the text data comprises assigning an attribute to at least one identified pathology or condition.

10. A medical data processing apparatus according to claim 4, wherein the comparing of the model performance at the first time period and the model performance at the reference time period comprises comparing at least one statistical measure of performance.

11. A medical data processing apparatus according to claim 10, wherein the at least one statistical measure of performance and/or model drift score comprises, represents or is derived from at least one of F1, Precision, Recall, AUROC, Brier score or other scoring function or comparison between distributions.

12. A medical data processing apparatus according to claim 4, wherein the performance of the model at the reference time period is determined by comparing outputs of the model to ground truth data, and wherein the ground truth data comprises annotations of, or findings derived from, medical image data by a human expert, and/or the ground truth data for the reference time period comprises reference proxy ground truth data obtained by processing further text data for the reference time period.

13. A medical data processing apparatus according to claim 4, wherein the comparing of the performance of the model comprises excluding pathologies that are not mentioned in ground truth data and/or proxy ground truth data in determining the performance of the model.

14. A medical data processing apparatus according to claim 1, wherein the processing circuitry is configured to compare distributions of the input data and/or the reference data or findings or representations obtained from the input data and/or reference data, over time thereby to determine a measure of data drift over time.

15. A medical data processing apparatus according to claim 1, wherein the processing circuitry is configured to monitor variation over time for the output obtained from the input and/or for the at least one further finding obtained from processing of further medical data, thereby to obtain a measure of concept drift.

16. A medical data processing apparatus according to claim 15, wherein at least one of:
the measure of concept drift represents a likelihood of obtaining a given output, or finding, for a given input, or further medical data; or
the input and/or the further medical data used to obtain the measure of concept drift are either the same or different for different times.

17. A medical data processing apparatus according to claim 3, wherein the model drift, data drift and/or concept drift are determined for selected sub-sets of the input data and/or the further medical data corresponding to sub-sets of a set of patients, thereby to determine measures of model drift, data drift and/or concept drift that are specific to different sub-sets of patients.

18. A medical data processing apparatus according to claim 17, wherein the sub-sets of patients are selected based upon demographic, medical or other patient information.

19. A computer-implemented method for determining performance of a trained model, comprising:
providing to the model an input comprising medical data, from an imaging modality, in response to which the model provides an output comprising at least one finding;
obtaining at least one further finding from processing of further medical data; and
comparing the at least one finding and the at least one further finding thereby to determine performance of the model.

20. A medical data processing method according to claim 19, wherein at least one of:
the medical data comprises medical image data;
the further medical data comprises text data
